(19) United States
(12) Patent Application Publication
(10) Pub. No.: US 2022/0054393 A1
(43) Pub. Date: Feb. 24, 2022

(54) COSMETIC COMPOSITION FOR KERATIN FIBRES COMPRISING A FATTY ACID ESTER

(71) Applicant: L'OREAL, Paris (FR)

(72) Inventors: Mohamed BOULARAS, Chevilly Larue (FR); Philippe ILEKTI, Chevilly Larue (FR)

(73) Assignee: L'OREAL, Paris (FR)

(21) Appl. No.: 17/416,204

(22) PCT Filed: Dec. 20, 2019
(86) PCT No.: PCT/EP2019/086690

§ 371 Date: Jun. 18, 2021

(30) Foreign Application Priority Data
Dec. 21, 2018 (FR) 1873837

(51) Int. Cl. A61K 8/85; A61K 8/37; A61K 8/36; A61K 8/73; A61Q 1/10

(52) U.S. Cl. CPC A61K 8/85; A61K 8/37; A61K 2800/43; A61K 8/731; A61Q 1/10; A61K 8/361

(57) ABSTRACT

The present invention relates to a composition comprising: (1) at least one linear fatty acid monoester of formula (I): R₁—O—R₂, in which R₁ and R₂ are linear and saturated and have, independently of one another, a number of carbon atoms greater than or equal to 20, with R₁ representing an acyl radical, and R₂ representing an alkyl radical, (2) at least one fatty acid having from 14 carbon atoms to less than 20 carbon atoms, (3) at least one base capable of at least partially neutralizing at least one fatty acid (2), (4) at least one fatty alcohol, (5) at least one semi-crystalline polymer, (6) at least one hydrophilic gelling agent of polysaccharide type, and (7) water.

The present invention relates to the field of caring for and/or making up keratin materials, and is directed towards proposing compositions more particularly intended for making up the eyelashes or the eyebrows.

The term “keratin materials” is preferably intended to mean human keratin materials, especially keratin fibres.

The term “keratin fibres” is in particular intended to mean the eyelashes and/or the eyebrows, and preferably the eyelashes. For the purposes of the present invention, this term “keratin fibres” also extends to synthetic false eyelashes.

### TECHNICAL FIELD

In general, compositions intended for making up keratin fibres, for example the eyelashes, aim to densify the thickness and the visual perception of the eyelashes and ultimately the gaze. These mascaras are described as aqueous or else cream mascaras, when they are formulated in an aqueous base, and anhydrous mascaras when they are formulated as a dispersion in an organic solvent medium.

A great diversity of cosmetic effects can be provided by the application of a mascara to keratin fibres and in particular the eyelashes, for instance a volumising, lengthening, thickening and more particularly charging makeup effect.

These effects are mostly adjusted through the amount and nature of the particles and most particularly those of the waxes present in the mascaras. In general, mascaras in fact have a significant amount of wax(es) and in particular from 10% to 35% by weight of wax(es), more generally from 15% to 30% by weight, relative to the total weight thereof.

For obvious reasons, improving the textures of mascara which condition the manifestation of one or more makeup effects is a constant preoccupation of those producing cosmetic formulations.

Moreover, the specific effects associated with a particular formulation, for example charging and moreover providing excellent separation of the eyelashes made up, are expected to be reproduced virtually identically by all the production batches of one and the same formulation.

In order to meet these expectations and/or objectives, it is therefore necessary to be capable of precisely adjusting the texture of a mascara and of reproducing it as faithfully as possible with batches that are not necessarily produced at the same time but which are identical in terms of ingredients and must therefore provide makeup effects that are in theory also identical.

### PRIOR ART

However, as specified above, most of the mascaras currently available are formulated with a significant amount of waxes. In point of fact, as detailed in the document Ullmann's Encyclopedia of Industrial Chemistry 2015, Wiley-VCH Verlag GmbH & Co. KGaA, Weinheim 10.1002/14356007.a28.pub2, most waxes are not constituted of a single chemical compound, but are instead complex mixtures. They may be mixtures of oligomers and/or of polymers which, in many cases, also have varied molar masses, varied molar mass distributions and also varied degrees of branching. Thus, a polar wax is conventionally made up of a mixture of alkanes, fatty alcohols and fatty esters, the fatty-chain length of which varies according to the melting point.

It is therefore very difficult for the producers of these waxes to guarantee a rigorously identical composition for all production batches. More specifically, there may be, between several production batches of one and the same wax, a variability in terms of the chemical nature of some of its constituent compounds. Likewise, the proportionality of some of its constituent compounds may also vary between production batches.

For obvious reasons, these variabilities have a not insignificant impact on the properties of the wax and therefore on those of the mascara incorporating this wax in significant amount. Thus, two mascara formulations of identical composition and therefore produced from one and the same conventional wax in the same amount, can nevertheless diverge in terms of rheological properties and therefore of texture, if they were produced from two separate production batches of this wax.

### PRESENTATION OF THE INVENTION

Consequently, the use of conventional waxes, in particular in significant amount, in mascara compositions does not make it possible to guarantee for users the reproduction of finely adjusted and totally identical rheological properties in all the mascara specimens of one and the same composition.

The present invention aims precisely to provide mascara compositions which have dispensed with this limitation.

Thus, a first objective of the present invention is to obtain mascara compositions of which the texturing properties are finely adjustable and reproducible.

Another objective of the present invention is to provide a mascara architecture produced using a predominant weight proportion of single-component ingredients. The predominant use of single-component ingredients advantageously makes it possible to dispense with the risk of composition variability that may exist between several production batches of a multicomponent ingredient and therefore to dispense with the risk of its impact on the final properties of the mascara.

Another objective of the present invention is to provide a mascara architecture which makes it possible to significantly reduce or even dispense with the use of waxes, but which nevertheless remains very satisfactory in terms of makeup effect.

Thus, another objective of the invention is to provide consumers with compositions, especially cosmetic compositions, and in particular mascaras, that are compatible with a long playtime while at the same time making it possible to preserve the separation of the eyelashes.

Another objective of the invention is to also provide consumers with compositions which have a creamy texture.

Finally, an objective of the invention is to meet, for the most part, the above-mentioned subjects while at the same time providing the composition with great stability.

The term “stable composition” is intended to mean that the composition remains usable as makeup after storage for two months at a temperature of 45° C. and that it retains its pleasantness and its sensory signature on application. More specifically, a “stable composition” according to the invention has an acceptable change in viscosity, that is to say that the difference between the initial viscosity and the viscosity after storage for two months at a temperature of 45° C. remains a less than 10 Pa·s.

Unexpectedly, the inventors have noted that it is possible to significantly or even totally dispense with waxes as texturing agent in formulations for making up and/or caring for keratin fibres and therefore with the limitations mentioned above, with the proviso of combining very specific compounds in such formulations.

### SUMMARY OF THE INVENTION

Thus, according to a first of its aspects, the present invention relates to a composition, preferably a cosmetic composition, for caring for and/or making up keratin materials, in particular the eyelashes and/or the eyebrows, comprising:

(1) at least one linear fatty acid monoester of formula (I):

[Chem 1]

R₁—O—R₂ (I)

in which R₁ and R₂ are linear and saturated and have, independently of one another, a number of carbon atoms greater than or equal to 20, with R₁ representing an acyl radical, and R₂ representing an alkyl radical,
(2) at least one fatty acid having from 14 carbon atoms to less than 20 carbon atoms,
(3) at least one base capable of at least partially neutralizing at least one fatty acid (2),
(4) at least one fatty alcohol preferably chosen from C₁₄-C₃₀ fatty alcohols, better still chosen from the linear and saturated C₁₄-C₂₄, even better still C₁₄-C₂₀, fatty alcohols,
(5) at least one semi-crystalline polymer,
(6) at least one hydrophilic gelling agent of polysaccharide type, and
(7) water.

Unexpectedly, the inventors have in fact noted that the formulation in an aqueous medium of at least one linear fatty acid monoester (1) in combination with at least one neutralized form of a fatty acid having from 14 carbon atoms to less than 20 carbon atoms, at least one fatty alcohol preferably chosen from C₁₄-C₃₀ fatty alcohols, better still chosen from the linear and saturated C₁₄-C₂₄, even better still C₁₄-C₂₀, fatty alcohols (4), at least one semi-crystalline polymer (5) and at least one hydrophilic gelling agent of polysaccharide type (6), makes it possible to obtain compositions of which the texture can be finely adjusted and guaranteed in terms of reproducibility.

As emerges from what follows, these new compositions are advantageous in several respects.

First of all, the compounds (1) to (6) required according to the invention are of synthetic origin and, in this respect, of increased purity compared for example with a natural wax.

They are also, as individualized compounds, single-component compounds or compounds having a well-defined number of components, as opposed to the majority of conventional waxes which are often multicomponent or even have an indefinite number of compounds, such as natural waxes and some synthetic waxes.

These two specificities are particularly advantageous since they make it possible to dispense with a risk of variability with regard to their respective compositions.

As emerges from the examples below, the compositions in accordance with the invention and based on the use of the compounds (1) to (6) as texturing agent prove to be very satisfactory in terms of makeup effects.

Thus, compositions according to the invention may have a creamy texture which proves to be finely adjustable by virtue of the use of the required combination according to the invention.

The obtaining of these properties is conditioned by the use of the compounds (1) to (6) and advantageously does not therefore require the additional presence of waxes, in particular in significant amount.

Thus, the compositions according to the invention advantageously comprise less than 5% of waxes as defined below.

The term “wax” is intended to mean lipophilic compounds, which are solid at ambient temperature (20° C.) and at atmospheric pressure (760 mmHg), with a reversible solid/liquid change of state, which have a melting point of greater than or equal to 40° C., which may range up to 120° C.

For the purposes of the invention, the waxes to which this abovementioned amount limitation relates are distinct from those capable of being embodied by the component which is a monoester of fatty acid(s) (1) and fatty alcohol (4) required according to the invention.

Finally, the manifestation of the makeup effects provided by the combination of the compounds (1) to (6) is not acquired to the detriment of the stability of the compositions.

According to another of its aspects, the present invention relates to a process, in particular a cosmetic process, for caring for and/or making up keratin materials, in particular the eyelashes and/or the eyebrows, comprising at least one step consisting in applying, to said keratin materials, in particular the eyelashes and/or eyebrows, a composition in accordance with the invention.

### DETAILED DESCRIPTION

Fatty Acid Monoester (1)

A composition according to the invention comprises at least one linear fatty acid monoester.

A composition according to the invention may comprise at least 5.0% by weight, preferably at least 6.0% by weight, better still at least 7.0% by weight of linear fatty acid monoester(s), relative to the total weight of the composition.

According to one particularly preferred embodiment of the invention, the fatty acid monoester(s) are present in the composition in a content ranging from 6.0% to 35.0% by weight, preferably from 7.0% to 30.0% by weight, or even preferably from 8.0% to 28.0% by weight, relative to the total weight of the composition.

The linear fatty acid monoester(s) (1) in question according to the invention correspond to formula (I) below:

[Chem 2]

R₁—O—R₂ (I)

in which R₁ and R₂ are linear and saturated and have, independently of one another, a number of carbon atoms greater than or equal to 20, with R₁ representing an acyl radical, and R₂ representing an alkyl radical.

This or these fatty acid monoester(s) is (are) used during the preparation of a composition according to the invention, in an individualized form or in the form of a mixture comprising exclusively linear fatty acid monoesters of formula (I).

In one preferred embodiment, the fatty acid monoester(s) has (have) a melting point greater than 50° C.

The melting point may be measured by any known method and in particular using a differential scanning calorimeter (DSC).

According to one preferred embodiment of the invention, the acyl and alkyl radicals representing respectively R₁ and R₂ are chosen in such a way that the compound (I) is solid at a temperature of less than or equal to 30° C.

According to one particularly preferred embodiment of the invention, R₁ and R₂ are, respectively, acyl and alkyl radicals having a number of carbon atoms ranging from 20 to 30, preferably from 20 to 24.

According to one particularly preferred embodiment, R₁ and R₂ are, respectively, acyl and alkyl radicals having the same number of carbon atoms.

In particular, the fatty acid monoester according to the invention is chosen from arachidyl arachidate and behenyl behenate.

According to one particularly preferred embodiment of the invention, the linear fatty acid monoester is a behenyl behenate.

A behenyl behenate suitable for the composition according to the invention may in particular be Kester Wax K-72 sold by the company Koster Keunen, DUB BB sold by the company Stearinerie Dubois, or Dermowax BB sold by Alzo.

Ionic Surfactant

As specified above, the fatty acid monoester(s) (1) used according to the invention are combined with at least one ionic surfactant resulting from the neutralization of a fatty acid (2) comprising from 14 to less than 20 carbon atoms by a base (3).

Fatty Acid (2)

The fatty acid according to the invention comprises from 14 to less than 20 carbon atoms. According to one preferred embodiment of the invention, the fatty acid comprises from 16 to less than 20 carbon atoms. According to one particularly preferred embodiment, the number of carbon atoms ranges from 16 to 18.

In particular, the fatty acid(s) according to the invention is (are) chosen from linear fatty acids, saturated fatty acids and mixtures thereof.

According to one particularly advantageous embodiment of the invention, the fatty acid of the ionic surfactants is linear and saturated.

According to one particular embodiment of the invention, the fatty acid(s) is (are) chosen from palmitic acid, stearic acid and mixtures thereof, and preferably comprises at least stearic acid having the INCI name STEARIC ACID.

Thus, according to another embodiment of the invention, the composition uses, as fatty acid (2), a mixture of C₁₆-C₁₈ fatty acids, preferably a mixture of fatty acids having 16 carbon atoms, such as palmitic acid, and of fatty acids having 18 carbon atoms, such as stearic acid.

A preferred stearic acid suitable for the invention is, for example, Stearic Acid 1850 sold by the company Southern Acids.

A composition according to the invention may comprise at least 3.0% by weight of fatty acid(s) (2) relative to the total weight of the composition

According to one preferred embodiment of the invention, the fatty acid is present in a content ranging from 3.5% to 20.0% by weight, preferably from 4.0% to 20.0% by weight, better still from 4.5% to 15.0% by weight, even better still from 5.0% to 15.0% by weight of fatty acid (2), in particular of stearic acid, relative to the total weight of the composition.

As mentioned above, this fatty acid is used in an ionic form generated via its interaction with an organic base. This base is used at an amount sufficient to be capable of at least partially neutralizing at least one fatty acid (2).

Base (3)

The composition according to the invention comprises at least one base. This base may be organic or inorganic.

According to a first variant, the base is at least one organic base.

This organic base is chosen from ammonium and its amine and amino alcohol derivatives.

Preferably, the base of organic origin is chosen from alkanoalamines such as monoethanolamine, diethanolamine, triethanolamine, triisopropanolamine, aminomethylpropanol, tromethamine and aminomethylpropanediol, and mixtures thereof. According to one particular embodiment, the base is a primary (poly)hydroxyalkylamine. The term “primary (poly)hydroxyalkylamine” is intended to mean in particular a primary dihydroxyalkylamine, the term “primary” being intended to mean a primary amine function, i.e. —NH₂, and the alkyl group being a linear or branched C₁-C₈, preferably C₄ branched, hydrocarbon-based chain, such as 1,3-dihydroxy-2methylpropyl. The primary (poly)hydroxyalkylamine is preferably 1,3-dihydroxy-2-methyl-2-propylamine (also known as aminomethylpropanediol or AMPD).

According to one preferred embodiment of the invention, the base of organic origin is chosen from triethanolamine, aminomethylpropanol, aminomethylpropanediol, and mixtures thereof, and is preferably aminomethylpropanediol.

Such an aminomethylpropanediol suitable for the invention is, for example, AMPD Ultra PC sold by the company Angus (Dow Corning).

According to a second variant, the base is at least one inorganic base.

This inorganic base is chosen from alkali metal hydroxides.

Preferably, the inorganic base is chosen from sodium hydroxide, potassium hydroxide, and mixtures thereof.

According to one preferred embodiment of the invention, the inorganic base is sodium hydroxide.

The amount of base is adjusted so as to obtain sufficient neutralization to confer an effective ionicity on the associated fatty acid (2).

Preferably, the base is present in an amount sufficient to neutralize some or all of the carboxylic functions of the fatty acid(s) (2) comprising from 14 to less than 20 carbon atoms. In one preferred embodiment, the amount of base is such that it is capable of neutralizing all the acid functions of the fatty acid (2).

For example, the composition according to the invention may comprise at least 0.1% by weight, better still at least 0.15% by weight, of base(s), relative to the total weight of the composition.

According to one preferred embodiment of the invention, the base is present in a content ranging from 0.2% to 3.0% by weight, preferably from 0.3% to 2.0% by weight of base(s), in particular of sodium hydroxide or of aminomethylpropanediol, relative to the total weight of the composition according to the invention.

According to one particular embodiment of the invention, the compositions according to the invention contain an ionic surfactant resulting from the total neutralization of stearic acid by sodium hydroxide.

According to another particular embodiment of the invention, the compositions according to the invention contain an ionic surfactant resulting from the total neutralization of stearic acid by aminomethylpropanediol.

The fatty acid (2) and the base (3) making up the neutralized ionic surfactant according to the invention can be introduced into the composition in the form of one and the same commercial material, or one after the other in the form of two distinct commercial materials. Preferably, the fatty acid (2) and the base (3) will be introduced into the composition in the form of two distinct commercial materials.

Fatty Alcohol (4)

The compositions according to the invention also comprise at least one fatty alcohol. A composition can therefore comprise a single fatty alcohol according to the invention or several distinct fatty alcohols.

If several distinct fatty alcohols are present, they can be added separately during the preparation of the composition and the mixture thereof can then be formed in situ. They can also be used in the form of a mixture which is already commercially available and in which the weight proportion and the degree of purity of each of the fatty alcohols are controlled. In other words, the composition of these mixtures is faithfully reproducible, as opposed to mixtures of fatty alcohols generated via synthesis from starting materials derived from complex mixtures.

The fatty alcohol(s) is (are) in particular chosen from linear or branched, saturated or unsaturated C₁₄-C₃₀, preferably C₁₄-C₂₄, and even better still C₁₄-C₂₀, fatty alcohols.

The fatty alcohol(s) is (are) in particular chosen from linear and saturated C₁₄-C₃₀ fatty alcohols, preferably linear and saturated C₁₄-C₂₄ fatty alcohols, and better still linear and saturated C₁₄-C₂₀ fatty alcohols.

According to one particularly preferred embodiment, the fatty alcohol is in the form of a mixture of several different fatty alcohols, and preferably is a mixture of several linear and saturated C₁₄-C₃₀, better still C₁₄-C₂₄, even better still C₁₄-C₂₀, fatty alcohols.

Preferably, the fatty alcohol according to the invention is chosen from (C₁₆) cetyl alcohol, (C₁₈) stearyl alcohol and mixtures thereof (also known as “cetearyl alcohol”). Preferentially, the fatty alcohol according to the invention is a mixture of cetyl alcohol and stearyl alcohol. Such a mixture is in particular sold under the name Lannette O OR/MB by the company BASF.

According to one preferred embodiment, the fatty alcohol is solid at ambient temperature.

The fatty alcohol is present in the compositions of the invention in amounts ranging from 1.0% to 20.0% by weight, relative to the total weight of the composition, preferably from 2.0% to 15.0% by weight, and even more particularly from 3.0% to 10.0% by weight, relative to the total weight of the composition.

Semi-Crystalline Polymer (5)

The composition according to the invention comprises at least 2.0% by weight, preferably from 3.0% to 20% by weight, better still from 4.0% to 15.0% by weight, even better still from 5.0% to 15.0% by weight of semi-crystalline polymer(s), relative to the total weight of the composition.

For the purposes of the invention, the term “polymers” is intended to mean compounds comprising at least two repeating units, preferably at least three repeating units and more especially at least ten repeating units.

For the purposes of the invention, the term “semi-crystalline polymer” is intended to mean polymers comprising a crystallizable portion and an amorphous portion and having a first-order reversible change of phase temperature, in particular of melting (solid-liquid transition). The crystallizable portion is preferably a chain that is lateral (or a chain that is pendent) relative to the backbone.

Besides the crystallizable chains or blocks, the blocks of the polymers are amorphous.

For the purposes of the invention, the term “crystallizable chain or block” is intended to mean a chain or block which, if it were alone, would change from the amorphous state to the crystalline state reversibly, depending on whether the temperature is above or below the melting point. For the purposes of the invention, a chain is a group of atoms, which are pendent or lateral relative to the polymer backbone.

When the crystallizable portion is a chain that is pendent relative to the backbone, the semi-crystalline polymer may be a homopolymer or a copolymer.

Preferably, the semi-crystalline polymer has an organic structure.

The term “organic compound” or “having an organic structure” is intended to mean compounds containing carbon atoms and hydrogen atoms and optionally heteroatoms such as S, O, N or P, alone or in combination.

The semi-crystalline polymer(s) according to the invention are solids at ambient temperature (25° C.) and atmospheric pressure (760 mmHg), the melting point of which is greater than or equal to 30° C.

The melting point values correspond to the melting point measured using a differential scanning calorimeter (D.S.C.) such as the calorimeter sold under the name DSC 30 by the company Mettler, with a temperature rise of 5° C. or 10° C. per minute (the melting point in question is the point corresponding to the temperature of the most endothermic peak in the thermogram).

According to one particular embodiment, the semi-crystalline polymer(s) used in the composition of the invention have a melting point Mp of less than 95° C., preferably less than 85° C. The semi-crystalline polymer(s) can thus have a melting point Mp ranging from 30° C. to 95° C. and preferably from 40° C. to 85° C. This melting point is preferably a first-order change of state temperature.

According to the invention, the semi-crystalline polymers are advantageously soluble in the fatty phase, especially to at least 1.0% by weight, at a temperature that is higher than their melting point. Preferably, the polymer backbone of the semi-crystalline polymers is soluble in the fatty phase at a temperature above their melting point.

Preferably, the crystallizable blocks or chains of the semi-crystalline polymers represent at least 30% of the total weight of each polymer and better still at least 40%.

When the semi-crystalline polymers of the invention are crystallizable block polymers, they can be block or multiblock copolymers. They may be obtained by polymerizing a monomer bearing reactive (or ethylenic) double bonds or by polycondensation. When the semi-crystalline polymers of the invention are polymers bearing crystallizable side chains, these side chains are advantageously in random or statistical form.

Preferably, the semi-crystalline polymers of the invention are of synthetic origin.

According to one preferred embodiment, the semi-crystalline polymer is chosen from homopolymers and copolymers comprising units resulting from the polymerization of one or more monomers bearing one or more crystallizable hydrophobic side chain(s).

The semi-crystalline polymers that can be used in the invention may be chosen in particular from homopolymers or copolymers, in particular those bearing at least one crystallizable side chain, such as those described in document U.S. Pat. No. 5,156,911.

In one preferred embodiment, the crystallizable side chain(s) are hydrophobic.

These homopolymers or copolymers can result:

from the polymerization, in particular the radical polymerization, of one or more monomers containing double bond(s) which is (are) reactive, or which is (are) ethylenic, with respect to a polymerization, namely a vinyl, (meth)acrylic or allylic group,

from the polycondensation of one or more monomers bearing co-reactive groups (carboxylic acid, sulfonic acid, alcohol, amine or isocyanate), for instance polyesters, polyurethanes, polyethers or polyureas.

In general, the crystallizable units (chains or blocks) of semi-crystalline polymers according to the invention originate from monomer(s) containing crystallizable block(s) or chain(s), used for manufacturing semi-crystalline polymers. These polymers are preferably chosen especially from homopolymers and copolymers resulting from the polymerization of at least one monomer containing crystallizable chain(s) that may be represented by the following formula:

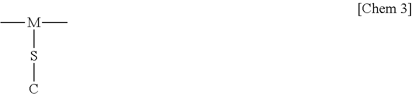

with M representing an atom of the polymer backbone, C representing a crystallizable group, and S representing a spacer, the crystallizable “—S—C” chains being hydrocarbon-based aliphatic or aromatic chains, comprising saturated or unsaturated, hydrocarbon-based alkyl chains, which are for example C₁₀-C₄₀, preferably C₁₀-C₃₀. “C” represents in particular a linear or branched or cyclic (CH₂)ₙ group, with n being an integer ranging from 10 to 40. Preferably, “C” is a linear group. Preferably, “S” and “C” are different.

When the crystallizable chains are hydrocarbon-based aliphatic chains, they comprise hydrocarbon-based alkyl chains containing at least 10 carbon atoms and not more than 40 carbon atoms and better still not more than 30 carbon atoms. They are in particular aliphatic chains or alkyl chains containing at least 10 carbon atoms, and they are preferably C₁₀-C₄₀, preferably C₁₀-C₃₀, alkyl chains.

Preferably, the crystallizable chains are hydrocarbon-based aliphatic C₁₀-C₃₀ chains.

As examples of semi-crystalline homopolymers or copolymers containing crystallizable chain(s) that are suitable for the invention, mention may be made of those resulting from the polymerization of one or more of the following monomers: saturated alkyl (meth)acrylates with the alkyl group being C₁₀-C₃₀, N-alkyl(meth)acrylamides with the alkyl group being C₁₀ to C₃₀, vinyl esters containing alkyl chains with the alkyl group being C₁₀ to C₃₀, vinyl ethers containing alkyl chains with the alkyl group being C₁₀ to C₃₀, C₁₀ to C₃₀ alpha-olefins such as, for example, octadecene, para-alkylstyrenes with an alkyl group containing from 10 to 30 carbon atoms, and mixtures thereof.

When the polymers result from a polycondensation, the hydrocarbon-based crystallizable chains as defined above are borne by a monomer that may be a diacid, a diol, a diamine or a diisocyanate.

When the semi-crystalline polymers which are the subject of the invention are copolymers, they additionally contain from 0 to 50% of groups Y which is a polar monomer, a non-polar monomer or a mixture of the two.

When Y is a polar monomer, it is either a monomer bearing polyoxyalkylene (in particular oxyethylene and/or oxypropylene) groups, a hydroxyalkyl (meth)acrylate, such as hydroxy ethyl acrylate, (meth)acrylamide, an N-alkyl(meth)acrylamide, an NN-dialkyl(meth)acrylamide, for instance NN-diisopropylacrylamide or N-vinylpyrolidone (NVP), N-vinylcaprolactam, or a monomer bearing at least one carboxylic acid group, such as (meth)acrylic, crotonic, itaconic, maleic or fumaric acids, or bearing a carboxylic acid anhydride group, such as maleic anhydride, and mixtures thereof.

When Y is a non-polar monomer, it may be an ester of the linear, branched or cyclic alkyl (meth)acrylate type, a vinyl ester, an alkyl vinyl ether, an α-olefin, styrene or styrene substituted with a C₁ to C₁₀ alkyl group, for instance α-methylstyrene, or a macromonomer of the polyorganosiloxane type comprising a vinyl unsaturation.

Preferably, the semi-crystalline polymers containing one or more crystallizable side chain(s) are alkyl (meth)acrylate or alkyl(meth)acrylamide homopolymers with an alkyl group as defined above, and in particular of C₁₀-C₃₀, copolymers of these monomers with a hydrophilic monomer preferably of different nature from (meth)acrylic acid, for instance N-vinylpyrrolidone or hydroxyethyl (meth)acrylate, and mixtures thereof.

It is also possible to use the semi-crystalline polymers obtained by copolymerization of behenyl acrylate and of acrylic acid or of N-vinylpyrrolidone, as described in document U.S. Pat. No. 5,519,063.

Advantageously, the semi-crystalline polymer(s) containing one or more crystallizable side chain(s) have a weight-average molecular weight Mw ranging from 5000 g/mol to 1 000 000 g/mol, preferably from 10 000 g/mol to 800 000 g/mol, preferentially from 15 000 g/mol to 500 000 g/mol, and more preferably from 80 000 g/mol to 200 000 g/mol.

According to one particular embodiment of the invention, the semi-crystalline polymer may be chosen from homopolymers and copolymers resulting from the polymerization of at least one monomer containing one or more crystallizable side chain(s) chosen from saturated C₁₀ to C₃₀ alkyl (meth)acrylates, which may be represented by the formula below:

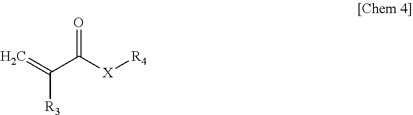

in which R₃ is H or CH₃, R₄ represents a C₁₀ to C₃₀ alkyl group and X represents O.

According to one more particular embodiment of the invention, the semi-crystalline polymer results from the polymerization of monomers containing one or more crystallizable side chain(s), chosen from saturated C₁₀ to C₃₀ alkyl (meth)acrylates.

The semi-crystalline polymers comprising crystallizable side chains can be chosen from the copolymers resulting from the copolymerization of acrylic acid and C₁₀ to C₃₀ alkyl (meth)acrylate, in particular such as those described in U.S. Pat. No. 5,156,911.

The semi-crystalline polymers may in particular be those described in Examples 3, 4, 5, 7 and 9 of U.S. Pat. No. 5,156,911, and more particularly those obtained by the copolymerization:
of acrylic acid, of hexadecyl acrylate and of isodecyl acrylate in a 1/16/3 ratio,
of acrylic acid and of pentadecyl acrylate in a 1/19 ratio,
of acrylic acid, of hexadecyl acrylate and of ethyl acrylate in a 2.5/76.5/20 ratio,
of acrylic acid, of hexadecyl acrylate and of methyl acrylate in a 5/85/10 ratio,
of acrylic acid and of polyoctadecyl (meth)acrylate in a 2.5/97.5 ratio.

As a particular example of a semi-crystalline polymer that may be used in the composition according to the invention, mention may be made of the Intelimer® products from the company Landec described in the brochure “Intelimer® polymers”, Landec IP22 (Rev. 4-97). These polymers are in solid form at ambient temperature (25° C.). They bear crystallizable side chains and have the preceding formula (II). They are poly(C₁₀-C₃₀)alkyl acrylates, which are particularly suitable as semi-crystalline polymers that may be included in a composition in accordance with the present invention.

According to one particularly preferred embodiment of the invention, the semi-crystalline polymer(s) (5) according to the invention is (are) derived from a monomer containing a crystallizable chain, chosen from saturated C₁₀ to C₃₀ alkyl (meth)acrylates and more particularly from poly(stearyl acrylate)s, poly(behenyl acrylate)s, and mixtures thereof.

Preferably, the semi-crystalline polymers suitable for the invention are in particular poly(stearyl acrylate), in particular the product sold under the name Intelimer® IPA 13-1, from the company Air Products and Chemicals or Landec, which is a poly(stearyl acrylate) of which the melting point is equal to 49° C., or poly(behenyl acrylate), sold under the name Intelimer® IPA 13-6, from the company Air Products and Chemicals or Landec, which is a poly(behenyl acrylate) of which the melting point is equal to 65° C.

According to one particularly preferred embodiment, the semi-crystalline polymer is at least one poly(behenyl acrylate).

Examples of homopolymers or copolymers suitable as semi-crystalline polymers for the invention preferably comprise from 50% to 100% by weight of units resulting from the polymerization of one or more monomers bearing a crystallizable hydrophobic side chain.

### Hydrophilic Gelling Agent of Polysaccharide Type (6)

For the purposes of the present invention, the term “hydrophilic gelling agent” is intended to mean a compound capable of gelling the aqueous phase of the compositions according to the invention.

The gelling agent is hydrophilic and is therefore present in the aqueous phase of the composition.

The gelling agent may be water-soluble or water-dispersible.

As specified above, these gelling agents fall within the category of polysaccharides.

In general, the compounds of this type, that can be used in the present invention, are chosen from those which are in particular described in “Encyclopedia of Chemical Technology, Kirk-Othmer, Third Edition, 1982, volume 3, pp. 896-900, and volume 15, pp. 439-458”, in “Polymers in Nature, by E. A. McGREGOR and C. T. GREENWOOD, publisher John Wiley & Sons, Chapter 6, pp. 240-328, 1980”, in the book by Robert L. DAVIDSON entitled “Handbook of Water soluble gums and resins” published by McGraw Hill Book Company (1980) and Industrial Gums “Polysaccharides and their Derivatives, edited by Roy L. WHISTLER, Second Edition, publisher Academic Press Inc.”.

These polysaccharides can be chemically modified, in particular with urea or urethane groups, or by hydrolysis, oxidation, esterification, etherification, alkylation reaction, or by several of these modifications.

Preferably, these polysaccharides are non-ionic.

The polysaccharides that are suitable for the invention may most particularly be homopolysaccharides such as fructans, glucans, galactans and mannans or heteropolysaccharides such as hemicellulose.

The polysaccharides that are suitable for the invention may also be chosen from the polysaccharides of higher plants, such as homogeneous polysaccharides, and in particular cellulose compounds and derivatives thereof.

According to the invention, the term “cellulose compound” is intended to mean any polysaccharide compound having in its structure linear sequences of anhydroglucopyranose (AGU) residues linked together by β(1,4) glycosidic bonds. The repeat unit is the cellobiose dimer. The AGUs are in the chair conformation and have three hydroxyl functions: two secondary alcohols (in position 2 and 3) and one primary alcohol (in position 6). The polymers thus formed link together via intermolecular bonds of hydrogen bond type, thus conferring a fibrillar structure on the cellulose (approximately 1500 molecules per fibre).

The degree of polymerization differs enormously according to the origin of the cellulose; its value can range from a few hundred to a few tens of thousands.

Cellulose has the following chemical structure:

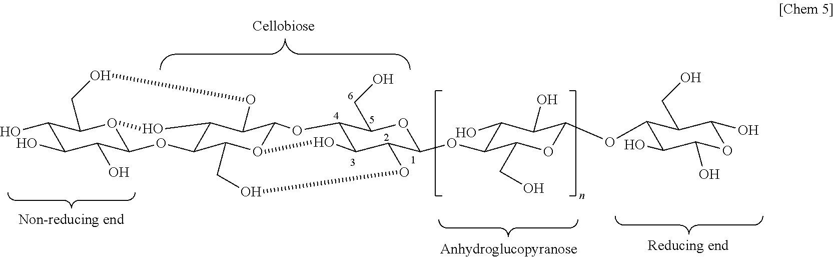

The hydroxyl groups of the cellulose can partially or totally react with various chemical reagents so as to give cellulose derivatives having specific properties. Among these derivatives, cellulose ethers, cellulose esters and cellulose ether esters are distinguished.

Among the cellulose derivatives that are most particularly suitable for the invention as hydrophilic gelling agent, mention may in particular be made of non-ionic cellulose ethers and esters. Thus, the hydrophilic gelling agent is preferably chosen from cellulose ethers, cellulose esters, and mixtures thereof.

Preferably, the hydrophilic gelling agent (6) is a hydroxyalkylcellulose and in particular is chosen from hydroxymethylcelluloses, hydroxyethylcelluloses and hydroxypropylcelluloses; and/or a hydroxyalkyl-alkyl cellulose mixed cellulose and in particular is chosen from hydroxypropylmethylcelluloses, hydroxyethylmethylcelluloses, hydroxyethylethylcelluloses, hydroxybutylmethylcelluloses and cetyl hydroxyethylcelluloses. Such compounds are in particular commercially available under the names Klucel® (hydroxypropylcellulose), Polysurf® and Natrosol® CS (cetyl hydroxyethylcellulose) sold by the company Ashland and also Cellosize® (hydroxyethylcellulose) sold by the company Dow Chemicals.

Advantageously, a composition according to the invention contains from 0.1% to 8% by weight of hydrophilic gelling agent (6) expressed by dry weight, in particular from 0.1% to 6% by weight, preferably between 0.5% and 2.5% by weight of hydrophilic gelling agent (6), relative to its total weight.

Water (7)

A composition according to the invention comprises water.

In particular, a composition according to the invention comprises at least 30.0% by weight, better still at least 40.0% by weight, or even a content of from 50% to 60% by weight of water, relative to the total weight of the composition.

In one preferred embodiment of the invention, the composition according to the invention comprises:

(1) at least one linear fatty acid monoester of formula (I):

[Chem 6]

R₁—O—R₂ (I)

in which R₁ and R₂ are linear and saturated and have, independently of one another, a number of carbon atoms ranging from 20 to 24, with R₁ representing an acyl radical, and R₂ representing an alkyl radical,
(2) at least one fatty acid chosen from stearic acid, palmitic acid and mixtures thereof,
(3) at least one base chosen from aminomethylpropanediol and alkali metal hydroxides,
(4) at least one linear and saturated fatty alcohol chosen from those which are C₁₄-C₂₀,
(5) at least one semi-crystalline polymer resulting from the polymerization of at least one monomer with crystallizable side chain(s),
(6) at least one cellulose derivative as hydrophilic gelling agent, and
(7) water.

In yet another particularly preferred embodiment, the composition according to the invention comprises:

(1) at least one linear fatty acid monoester of formula (I):

[Chem 7]

R₁—O—R₂ (I)

in which R₁ and R₂ are linear and saturated and have, independently of one another, a number of carbon atoms ranging from 20 to 24, with R₁ representing an acyl radical, and R₂ representing an alkyl radical,
(2) at least one fatty acid chosen from stearic acid, palmitic acid and mixtures thereof,
(3) at least one base chosen from aminomethylpropanediol and alkali metal hydroxides,
(4) at least one linear and saturated fatty alcohol chosen from those which are C₁₄-C₂₀,
(5) at least one poly(behenyl acrylate) as semi-crystalline polymer,
(6) at least one cellulose derivative, and
(7) water.

In yet another very particularly preferred embodiment, the composition according to the invention comprises:

(1) at least behenyl behenate as linear fatty acid monoester,
(2) at least one fatty acid chosen from stearic acid, palmitic acid and mixtures thereof,
(3) at least aminomethylpropanediol as base,
(4) at least one linear and saturated fatty alcohol chosen from those which are C₁₄-C₂₀,
(5) at least one poly(behenyl acrylate) as semi-crystalline polymer,
(6) at least cetyl hydroxyethylcellulose and/or hydroxyethylcellulose, and
(7) water.

In another very particularly preferred embodiment, the composition according to the invention comprises:

(1) at least behenyl behenate as linear fatty acid monoester,
(2) at least one fatty acid chosen from stearic acid, palmitic acid and mixtures thereof,
(3) at least one alkali metal hydroxide as base,
(4) at least one linear and saturated fatty alcohol chosen from those which are C₁₄-C₂₀,
(5) at least one poly(behenyl acrylate) as semi-crystalline polymer,
(6) at least hydroxyethylcellulose and/or cetyl hydroxyethylcellulose, and
(7) water.

Other Components

In addition to the abovementioned compounds, a composition according to the invention may of course comprise secondary ingredients.

a) Waxes

Thus, a composition according to the invention may also comprise a wax.

However, with regard to the objectives targeted by the present invention, the compositions according to the invention preferably comprise a reduced amount of wax and in particular less than 5% by weight, or even less than 3% by weight of waxes, relative to the total weight of said waxes.

As specified in the preamble, for the purposes of the invention, the term “waxes” is intended to mean lipophilic compounds, which are solid at ambient temperature (20° C.) and at atmospheric pressure (760 mmHg), with a reversible solid/liquid change of state, which have a melting point of greater than or equal to 40° C., which may range up to 120° C.

It is recalled that, for the purposes of the invention, the waxes to which the abovementioned amount limitation relates are distinct from those capable of being embodied by the component which is a monoester of linear fatty acid (1) and/or the fatty alcohol (4) component required according to the invention.

This limitation relates more particularly to waxes made up of complex mixtures which are in particular described in the document Ullmann's Encyclopedia of Industrial Chemistry 2015, Wiley-VCH Verlag GmbH & Co. KGaA.

Such waxes may in particular be natural, but may also be synthetic.

The term “natural” wax is intended to denote any wax which preexists naturally or which can be converted, extracted or purified from natural compounds which exist naturally.

Among natural waxes, mention may in particular be made of waxes termed fossil waxes, including those of petroleum origin, such as ozokerite, pyropissite, macrocrystalline waxes, also known as paraffins—including crude or gatsch waxes, gatsch raffinates, de-oiled gatsch, soft waxes, semi-refined waxes, filtered waxes, refined waxes—and microcrystalline waxes, termed microwaxes, including bright stock gatsch. The fossil waxes also contain lignite, also known as montan wax, or peat wax.

As natural waxes other than fossil waxes, mention may be made of animal and plant waxes.

As examples of plant waxes, mention may be made of carnauba wax, candelilla wax, ouricury wax, sugarcane wax, jojob waxa, Trithrinax campestris wax, raffia wax, alfalfa wax, wax extracted from Douglas fir, sisal wax, flax wax, cotton wax, Batavia dammar wax, cereal wax, tea wax, coffee wax, rice wax, palm wax, Japan wax, mixtures thereof and derivatives thereof.

As examples of animal waxes, mention may be made of beeswax, Ghedda wax, shellac, Chinese wax, lanolin, also known as wool wax, mixtures thereof and derivatives thereof.

These waxes are generally multicomponent. For example, natural beeswax is composed of approximately 70% of esters for the majority of monoesters (of fatty acid and of fatty alcohol), but also of hydroxy esters, of diesters and triesters and esters of sterols, and also of long-chain linear hydrocarbons, of free acids and of free alcohols. For obvious reasons, the weight portion of their ingredients and their degree of purity are difficult to guarantee from one production batch to another.

The term “synthetic” wax is intended to denote waxes of which the synthesis requires one or more chemical reactions carried out by a human being.

Among the synthetic waxes, semi-synthetic waxes and totally synthetic waxes can be distinguished. Synthetic waxes may be waxes obtained by means of a Fischer-Tropsch process, constituted for example of paraffins with a number of carbon atoms ranging from 20 to 50 or waxes of polyolefins, for example homopolymers or copolymers of ethylene, of propene or butene, or even longer-chain α-olefins. The latter can be obtained by thermomechanical degradation of polyethylene plastic, by the Ziegler process, by high-pressure processes, or else via processes catalyzed by metallocene species. These waxes may be crystallizable, partially crystallizable or amorphous. The abovementioned synthetic waxes are generally non-polar and can be chemically treated to obtain polar waxes, for example by one or more of the following reactions: air oxidation, grafting, esterification, neutralization by metal soaps, amidation, direct copolymerizations or addition reactions.

Again in that case, their composition may be constituted of a mixture of ingredients since the fatty-chain lengths are not well defined, thus forming a mixture of compounds having different fatty-chain lengths and for which it is difficult for manufacturers to guarantee perfect reproducibility from one production batch to another.

Consequently, the compositions according to the invention advantageously comprise less than 5% by weight, preferably less than 3% by weight of waxes, in particular of multicomponent natural or synthetic wax, relative to the total weight of the composition.

For the purposes of the invention, a multicomponent wax denotes a wax constituted of a mixture of several ingredients, either such that it exists naturally like natural waxes, or such that it is formed during the process of industrial synthesis of these materials.

In one particularly preferred embodiment of the invention, the composition is free of these waxes, in particular multicomponent natural or synthetic wax.

As specified above, the preferred texturing compounds according to the invention are by contrast and advantageously synthetic, single-component compounds, which are thus available in a form purified to more than 99%, like the compound (1) required according to the invention.

b) Other Surfactants

The composition according to the invention can comprise surfactants other than that formed by the neutralization of a fatty acid having from 14 carbon atoms to less than 20 carbon atoms (2) by the base (3), as co-surfactants.

However, according to one preferred embodiment of the invention, the composition comprises less than 5.0% by weight, preferably less than 2.0% by weight, relative to the total weight of the composition, of non-ionic surfactants, and in particular of non-ionic surfactants with an HLB, measured at 25° C., of greater than or equal to 7.

The term “HLB” (Hydrophilic Lipophilic Balance) is well known to those skilled in the art, and denotes the hydrophilic-lipophilic balance of a surfactant determined at 25° C. in the Griffin sense. The term “hydrophilic-lipophilic balance (HLB)” is intended to mean the equilibrium between the size and the strength of the hydrophilic group and the size and the strength of the lipophilic group of the surfactant. The HLB value according to Griffin is defined in J. Soc. Cosm. Chem. 1954 (volume 5), pages 249-256.

In one particularly preferred embodiment of the invention, the composition is free of non-ionic surfactants with an HLB, measured at 25° C., of greater than or equal to 7.

c) Liquid Fatty Phase

A composition according to the invention may also comprise a liquid fatty phase.

Such a liquid fatty phase is an organic phase that is liquid at ambient temperature (20° C.) and at atmospheric pressure (760 mmHg), non-aqueous and water-immiscible.

The liquid fatty phase may contain a non-volatile oil chosen from polar oils and non-polar oils, and mixtures thereof.

A composition of the invention may comprise from 1.0% to 20.0% by weight, from 2.0% to 12.0% by weight and preferentially from 2.0% to 8.0% by weight of non-volatile oil, relative to the total weight of the composition.

A composition according to the invention generally comprises less than 5.0% by weight, preferably less than 2.0% by weight of volatile oil(s), relative to the total weight of the composition. In one particularly preferred embodiment of the invention, the composition is free of volatile oils.

The term “volatile oil” is intended to mean an oil that can evaporate on contact with the skin in less than one hour, at ambient temperature (20° C.) and atmospheric pressure (760 mmHg). More specifically, a volatile oil has an evaporation rate ranging from 0.01 to 200 mg/cm²/min.

d) Colorant

A composition according to the invention, and in particular those intended for makeup, generally comprises at least one colorant such as pulverulent colorants, liposoluble dyes or water-soluble dyes.

The pulverulent colorants can be chosen from pigments and pearlescent agents.

The pigments may be white or coloured, mineral and/or organic, and coated or uncoated. Mention may be made, among inorganic pigments, of titanium dioxide, optionally surface treated, zirconium, zinc or cerium oxides, and also iron or chromium oxides, manganese violet, ultramarine blue, chromium hydrate and ferric blue. Among the organic pigments that may be mentioned are carbon black, pigments of D & C type and lakes based on cochineal carmine or on barium, strontium, calcium or aluminium.

The pearlescent agents may be chosen from white pearlescent pigments such as mica coated with titanium or with bismuth oxychloride, coloured pearlescent pigments such as titanium mica with iron oxides, titanium mica especially with ferric blue or chromium oxide, titanium mica with an organic pigment of the abovementioned type, and also pearlescent pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

Preferably, the composition according to the invention comprises a pulverulent colorant, preferably of pigment type, in particular metal oxides.

Preferably, said colorant is present in the composition in a content ranging from 2.0% to 25.0% by weight, preferably from 3.0% to 20.0% by weight, more particularly from 4.0% to 15.0% by weight, relative to the total weight of the composition.

e) Cosmetic Active Agents

As cosmetic active agents that may be used in the compositions according to the invention, mention may be made in particular of antioxidants, preservatives, fragrances, neutralizers, cosmetic active agents, for instance emollients, vitamins and screening agents, in particular sunscreens, and mixtures thereof.

These additives may be present in the composition in a content ranging from 0.01% to 15.0% of the total weight of the composition.

Of course, those skilled in the art will take care to choose the optional additional additives and/or their amounts in such a way that the advantageous properties of the composition according to the invention are not, or are not substantially, detrimentally affected by the envisaged addition.

Physical Characteristics

a) Solids Content

The composition according to the invention advantageously has a solids content at least equal to 42.0% by weight, and preferably at least 44.0% by weight, relative to the total weight of the composition, or even from 45.0% to 60.0% by weight, relative to the total weight of the composition.

For the purposes of the present invention, the “solids content” denotes the content of non-volatile matter.

The amount of solids content (abbreviated as SC) of a composition according to the invention is measured using a Halogen Moisture Analyzer HR 73 commercial halogen desiccator from Mettler Toledo. The measurement is performed on the basis of the weight loss of a sample dried by halogen heating, and thus represents the percentage of residual matter once the water and the volatile matter have evaporated off.

This technique is fully described in the machine documentation supplied by Mettler Toledo.

The measuring protocol is as follows:

Approximately 2 g of the composition, referred to hereinbelow as the sample, are spread out on a metal crucible, which is placed in the halogen desiccator mentioned above. The sample is then subjected to a temperature of 105° C. until a constant weight is obtained. The wet weight of the sample, corresponding to its initial weight, and the dry weight of the sample, corresponding to its weight after halogen heating, are measured using a precision balance.

The experimental error associated with the measurement is of the order of plus or minus 2%.

The solids content is calculated in the following manner:

Solids content (expressed as weight percentage)=100×(dry weight/wet weight). [Math 1]

b) Viscosity

A composition according to the invention is advantageously creamy at an ambient temperature of 20° C.

It is characterized by a viscosity of less than 40 Pa·s, or even preferably less than 35 Pa·s, or even less than 30 Pa·s, measured at an ambient temperature of 20° C. using an RM100® Rheomat.

Preferably, the viscosity of the compositions according to the invention ranges from 2.0 to 40.0 Pa·s, or even preferably from 2.5 to 35.0 Pa·s, more particularly from 3.0 to 30.0 Pa·s, measured at the ambient temperature of 20° C. using an RM100® Rheomat.

Such a viscosity is particularly advantageous since it is the most suitable for the device for applying mascara and since it enables easy use for the consumer for a charging result.

The composition may be produced via the known processes generally used in the cosmetics field.

The composition used according to the invention may be a makeup composition, a makeup base, in particular for keratin fibres, or base coat, a composition to be applied onto makeup, also known as topcoat, or else a composition for treating keratin fibres.

More especially, the composition according to the invention is a mascara.

Such compositions are especially prepared according to the general knowledge of those skilled in the art.

The expressions “between . . . and . . . ” and “ranging from . . . to . . . ” should be understood as being inclusive of the limits, unless otherwise specified.

In the description and the examples, the percentages are percentages by weight, unless otherwise indicated. The percentages are thus given by weight relative to the total weight of the composition. The ingredients are mixed in the order and under conditions that are readily determined by those skilled in the art.

The invention will now be described by means of examples which are present purely for illustrative purposes and should not be interpreted as examples that limit the invention.

### Example

Compositions of mascara type in accordance with the invention (Compositions 1 and 2) are prepared as described below.

Preparation of Phase A

The starting materials were carefully weighed out beforehand using a balance (precision=0.01 g). The ingredients of phase A were melted in a jacketed heating pan in which circulates an oil of which the temperature is controlled by means of a thermostatic oil bath. The setpoint temperature was fixed at 90° C. After total melting, the pigment was introduced with stirring using a Rayneri blender. Stirring was maintained until a homogeneous preparation was obtained.

Preparation of Phase B

The water was preheated in an electric kettle to 95° C. The preservatives and the base were introduced into the water in a beaker at a temperature of 80° C. with stirring using a Rayneri blender.

Emulsification of Phases A and B

Phase B was poured into phase A with stirring for 5 minutes at 90° C. using a Rayneri blender. Phase A+B was then cooled to ambient temperature with stirring.

Table 1 below details their respective compositions.

Each mascara thus obtained was transferred into a closed container to prevent it from drying out on contact with air. The stability of the samples (the state of dispersion of the fatty substances and of the pigments were evaluated under an optical microscope) and the viscosity were tested.

TABLE 1

| | | | Composition | Composition |
|---|---|---|---|---|
| | | | 1 according | 2 according |
| | | | to the | to the |
| | INCI name | Commercial reference | invention | invention |
| A | CETEARYL ALCOHOL | Lanette O OR/MB from | 4.00 | 4.00 |
| | (C₁₆-C₁₈) (compound (4)) | BASF | | |
| | BEHENYL BEHENATE (C₄₄) | Kester wax K-72 from | 15.00 | 15.00 |
| | (compound (1)) | Koster Keunen | | |
| | C₁₀₋₃₀ POLY(ALKYL | Intelimer IPA 13-6 | 6.00 | 6.00 |
| | ACRYLATE) (C₂₂) | Polymer from Air | | |
| | (compound (5)) | Products and Chemicals | | |
| | STEARIC ACID | Stearic acid 1850 from | 6.00 | 6.00 |
| | (compound (2)) | Southern Acids | | |
| | IRON OXIDES | Sunpuro Black Iron Oxide | 8.00 | 8.00 |
| | | C33-7001 from Sun | | |
| | CETYL HYDROXYETHYL- | Natrosol Plus 330 CS | 1.00 | / |
| | CELLULOSE (compound (6)) | from Ashland | | |
| | HYDROXYETHYL- | Cellosize Hydroxyethyl | / | 2.00 |
| | CELLULOSE (compound (6)) | Cellulose QP-4400H | | |
| | | Europe, CGR from | | |
| | | Amerchol (Dow Chemical) | | |
| B | PHENOXYETHANOL | Protectol PE CO from | 0.60 | 0.60 |
| | | BASF | | |
| | CHLORPHENESIN | Cosvat L from | 0.30 | 0.30 |
| | | KRAEBER | | |
| | CAPRYLYL GLYCOL | 199602 Hydrolite CG | 0.80 | 0.80 |
| | | from Symrise | | |
| | AMINOMETHYL | AMPD Ultra PC from | 1 | 1 |
| | PROPANEDIOL (AMPD) | Angus (Dow Chemical) | | |
| | (compound (3)) | | | |
| | WATER | | qsp. 100 | qsp. 100 |

| | | |
|---|---|---|
| Viscosity at 24 H (in Pa · s) | 15 | 4.7 |
| Viscosity after 2 months at 45° C. (in Pa · s) | 13.7 | 2.6 |

Compositions 1 and 2 exhibit performance levels that are appropriate for them to be used for the purposes of caring for and/or making up the eyelashes.

Their textures are pleasant on application. They have a long playtime. Thus, when they are applied to eyelashes, the eyelash separation is preserved even after a large number of brushstrokes.

The stability of the compositions was examined after a residence period of two months at 45° C. of compositions 1 and 2. The texture of the sample that was at 45° C. was then compared with respect to that of the sample that remained at ambient temperature.

A stable composition is a composition which retains its texture and its homogeneity and the application of which remains pleasant. Conversely, a composition is described as unstable if its texture has become much thicker, if phase separation occurs or if the composition is no longer pleasant on application.

All of the results obtained are reported in Table 2 below:

TABLE 2

| | Composition according to | Composition according to |
|---|---|---|
| | the invention | the invention |
| | 1 | 2 |
| Appearance to | Creamy texture | Creamy texture |
| the naked eye | | |
| Microscope | Fine and homogeneous | Fine and homogeneous |
| | emulsion | emulsion |
| Stability | Stable | Stable |

1: Composition, for caring for and/or making up keratin fibres, comprising:
(1) at least one linear fatty acid monoester of formula (I):
R₁—O—R₂ (I)
in which R₁ and R₂ are linear and saturated and have, independently of one another, a number of carbon atoms greater than or equal to 20, with R₁ representing an acyl radical, and R₂ representing an alkyl radical,
(2) at least one fatty acid having from 14 carbon atoms to less than 20 carbon atoms,
(3) at least one base capable of at least partially neutralizing at least one fatty acid (2),
(4) at least one fatty alcohol,
(5) at least one semi-crystalline polymer,
(6) at least one hydrophilic gelling agent of polysaccharide type, and
(7) water.

2: Composition according to claim 1, containing less than 5% by weight of waxes, relative to the total weight of said composition.

3: Composition according to claim 1, in which, in said formula (I) of the linear fatty acid monoester (1), R₁ and R₂ are, respectively, acyl and alkyl radicals having the same number of carbon atoms.

4. (canceled)

5: Composition according to claim 3, in which the linear fatty acid monoester (1) is chosen from arachidyl arachidate and behenyl behenate.

6: Composition according to claim 1, comprising at least 5.0% by weight of linear fatty acid monoester(s) (1) relative to the total weight of the composition.

7: Composition according to claim 1, in which the fatty acid(s) (2) is (are) chosen from linear fatty acids and saturated fatty acids, and mixtures thereof.

8: Composition according to claim 1, in which the fatty acid(s) (2) is (are) chosen from stearic acid and palmitic acid, and mixtures thereof.

9: Composition according to claim 1, comprising at least 3.0% by weight of fatty acid(s) (2), relative to the total weight of the composition.

10: Composition according to claim 1, in which the base (3) is at least one inorganic base.

11: Composition according to claim 1, in which the base (3) is at least one organic base.

12: Composition according to claim 1, comprising at least 0.1% by weight of base(s) (3), relative to the total weight of the composition.

13: Composition according to claim 1, comprising at least one fatty alcohol (4) chosen from (C₁₆) cetyl alcohol, (C₁₈) stearyl alcohol and mixtures thereof.

14: Composition according to claim 1, comprising at least from 1.0% to 20.0% by weight of fatty alcohol(s) (4), relative to the total weight of the composition.

15: Composition according to claim 1, in which the semi-crystalline polymer(s) (5) is (are) derived from a monomer containing a crystallizable chain, chosen from saturated C₁₀ to C₃₀ alkyl (meth)acrylates and mixtures thereof.

16: Composition according to claim 15, in which said semi-crystalline polymer (5) is at least one poly(behenyl acrylate).

17: Composition according to claim 1, comprising at least 2.0% by weight of semi-crystalline polymer(s) (5), relative to the total weight of the composition.

18: Composition according to claim 1, in which said hydrophilic gelling agent (6) is chosen from cellulose ethers, cellulose esters, and mixtures thereof.

19: Composition according to claim 1, in which said hydrophilic gelling agent (6) is a hydroxyalkylcellulose.

20: Composition according to claim 1, comprising from 0.1% to 8% by weight of dry matter of hydrophilic gelling agent(s) (6), relative to the total weight of the aqueous phase.

21: Composition according to claim 1, comprising at least 30% by weight of water (7), relative to the total weight of said composition.

22: Composition according to claim 1, comprising less than 5.0% by weight of volatile oil(s), relative to the total weight of the composition.

23: Composition according to claim 1, comprising less than 5.0% by weight relative to the total weight of the composition, of non-ionic surfactants with an HLB, measured at 25° C., of greater than or equal to 7.

24: Composition according to claim 1, comprising at least one pulverulent colorant.

25: Composition according to claim 1, comprising from 2.0% to 25.0% by weight of colorant, relative to the total weight of the composition.

26: Composition according to claim 1, in which the solids content is at least 42.0% by weight relative to the total weight of said composition.

27: Process for caring for and/or making up keratin materials, comprising applying, to said keratin materials a composition according to claim 1.
